(19) United States
(12) Patent Application Publication
(10) Pub. No.: US 2024/0207604 A1
(43) Pub. Date: Jun. 27, 2024

(54) Multi Channel Device for Directional Shaping of Electric Fields e.g., Tumor Treating Fields (TTFields)

(71) Applicant: Novocure GmbH, Root D4 (CH)

(72) Inventor: Richard DESLAURIERS, Woodbury, CT (US)

(73) Assignee: Novocure GmbH, Root D4 (CH)

(21) Appl. No.: 18/392,084

(22) Filed: Dec. 21, 2023

Related U.S. Application Data
(60) Provisional application No. 63/435,370, filed on Dec. 27, 2022.

(51) Int. Cl. A61N 1/04; A61N 1/36

(52) U.S. Cl. CPC A61N 1/0476; A61N 1/36002; A61N 1/36034

(57) ABSTRACT

The paths that alternating electric fields take through a body part can be controlled by providing at least eight electrically isolated signal generators, each of which is configured to apply electrical signals between a respective electrode element on one side of the target region and a respective electrode element on the opposite side of the target region. This replaces the prior art's single wide-cross-section electric field with eight independently controllable electric fields, each of which has a relatively narrow cross-section. Using these relatively narrow cross-section fields, either alone or in combination, can improve aiming of the electric field, which can in turn increase the field strength in the target region. Optionally, activation of these relatively narrow cross-section fields can be shifted in time to achieve steering of the overall electric field.

### CROSS REFERENCE TO RELATED APPLICATIONS

This application claims the benefit of U.S. Provisional Application 63/435,370, filed Dec. 27, 2022, which is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 150-200 kHz). See, for example, U.S. Pat. No. 7,565,205, which is incorporated herein by reference in its entirety. Alternating electric fields can also be used to treat medical conditions other than tumors. For example, as described in U.S. Pat. No. 10,967,167, which is incorporated herein by reference in its entirety, alternating electric fields can be used to increase the permeability of the blood brain barrier so that, e.g., chemotherapy drugs can reach the brain.

FIG. 1 depicts the prior art Optune® system, which delivers TTFields to patients via four transducer arrays 90 that are placed on the patient's skin near the tumor. The transducer arrays 90 are arranged in two pairs, with one pair of transducer arrays 90L, 90R positioned to the left and right of the tumor, and the other pair of transducer arrays 90A, 90P positioned anterior and posterior to the tumor. Each transducer array is connected via a multi-wire cable to an AC signal generator 95. The AC signal generator (a) sends an AC current through the anterior/posterior (A/P) pair of transducer arrays for 1 second, which induces an electric field with a first direction through the tumor; then (b) sends an AC current through the left/right (L/R) pair of arrays for 1 second, which induces an electric field with a second direction through the tumor; then repeats steps (a) and (b) for the duration of the treatment. Each transducer array includes a plurality (e.g., between 9 and 30) of electrode elements. And all of the electrode elements on any given transducer array are wired together (e.g., in series or in parallel).

FIG. 2 depicts a more detailed view of a set of four transducer arrays 90 in which the individual electrode elements that make up each of the transducer arrays are visible. In this example, each of the transducer arrays 90 includes nine round electrode elements that are supported by a self-adhesive substrate. In FIG. 2, each of the four transducer arrays 90 is positioned at a particular place on a patient's head.

Increasing the strength of alternating electric fields in the relevant target region (e.g., in a tumor such as a glioblastoma) will typically increase the efficacy of treatment. And it is often possible to increase the strength of the fields in the target region by shifting the positions of the transducer arrays 90 away from the exact positions depicted in FIG. 2. More specifically, shifting the positions of the transducer arrays 90 on the relevant body part (e.g., by a few centimeters in the up, down, right, left, front, and/or back directions) can change the paths of the alternating electric fields that travel through the body part. And changing the paths of the electric fields can increase in field strength in the target region, which can in turn increase the efficacy of treatment.

But it turns out that the degree of control over the paths of the electric fields that is achievable by shifting the positions of the transducer arrays is limited. And this in turn limits how much the field strength can be increased.

### SUMMARY OF THE INVENTION

One aspect of the invention is directed to a first apparatus for applying an electric field to a target region using at least eight first electrode elements positioned on a first side of the target region and at least eight second electrode elements positioned on a second side of the target region that is opposite to the first side of the target region. The first apparatus comprises at least eight electrically isolated first signal generators. Each of the first signal generators has a respective first control input. Each of the first signal generators is configured to, in response to a respective first control signal that arrives at the respective first control input, apply an electrical signal between a respective one of the at least eight first electrode elements and a respective one of the at least eight second electrode elements. The first apparatus also comprises a controller that is programmed to generate each of the first control signals.

In some embodiments of the first apparatus, each of the first signal generators is configured to, (a) in response to a respective first control signal that arrives at the respective first control input, apply a positive electrical signal between the respective one of the at least eight first electrode elements and the respective one of the at least eight second electrode elements, and (b) in response to a respective second control signal that arrives at the respective second control input, apply a negative electrical signal between the respective one of the at least eight first electrode elements and the respective one of the at least eight second electrode elements. Note that the use of the identifiers (a) and (b) does not imply that (a) must precede (b) in time.

Optionally, in the embodiments described in the previous paragraph, the controller may be further programmed to, for each of the first signal generators, apply the respective first control signal and the respective second control signal at respective times in an alternating sequence so that the respective first signal generator will generate an output that alternates between a positive electrical signal and a negative electrical signal.

Optionally, in the embodiments described in the previous paragraph, the controller may be further programmed to interpose a break in time between each first control signal and each second control signal.

In some embodiments of the first apparatus, the controller is further programmed to generate each of the first control signals in a sequence that causes each of the at least eight first signal generators to generate an output with a given waveform, wherein the output of each of the at least eight first signal generators is shifted in time with respect to an output of at least one other first signal generator. Optionally, in these embodiments, the controller may be further programmed to control the shifting in time to steer an electric field within the target region.

In some embodiments of the first apparatus, the controller is further programmed to generate each of the first control signals in a sequence that causes each of the at least eight first signal generators to generate an output with a given waveform, wherein the output of each of the at least eight first signal generators is shifted in time with respect to an output of at least one other first signal generator. The controller is also further programmed to generate each of the first control signals in a sequence that causes each of the at least eight first signal generators to generate an output that alternates between a positive electrical signal and a negative electrical signal.

Some embodiments of the first apparatus further comprise the at least eight first electrode elements and the at least eight second electrode elements.

Some embodiments of the first apparatus further comprise at least eight electrically isolated second signal generators. Each of the second signal generators has a respective second control input. And each of the second signal generators is configured to, in response to a respective second control signal that arrives at the respective second control input, apply an electrical signal between a respective one of at least eight third electrode elements and a respective one of at least eight fourth electrode elements. In these embodiments, the controller is further programmed to generate each of the second control signals.

Optionally, in the embodiments described in the previous paragraph, the controller may be further programmed to generate each of the first control signals in a sequence that causes each of the at least eight first signal generators to generate an output with a first given waveform, wherein the output of each of the at least eight first signal generators is shifted in time with respect to an output of at least one other first signal generator. And the controller is also programmed to generate each of the second control signals in a sequence that causes each of the at least eight second signal generators to generate an output with a second given waveform, wherein the output of each of the at least eight second signal generators is shifted in time with respect to an output of at least one other second signal generator.

Another aspect of the invention is directed to a first method for applying an electric field to a target region using at least eight first electrode elements positioned on a first side of the target region and at least eight second electrode elements positioned on a second side of the target region that is opposite to the first side of the target region. The first method comprises applying respective first electrical signals between respective ones of the at least eight first electrode elements and respective ones of the at least eight second electrode elements. The first electrical signals all have a given first waveform. Each of the first electrical signals is shifted in time with respect to at least one other first electrical signal. And each of the first electrical signals is electrically isolated from all other first electrical signals.

In some instances of the first method, the first electrical signals are applied shifted in time to steer an electric field within the target region. Optionally, in these instances, each of the first electrical signals alternates between a positive polarity and a negative polarity.

Some instances of the first method further comprise positioning the at least eight first electrode elements on the first side of the target region; and positioning the at least eight second electrode elements on the second side of the target region.

Some instances of the first method further comprise applying respective second electrical signals between respective ones of at least eight third electrode elements and respective ones of at least eight fourth electrode elements. The second electrical signals all have a given second waveform. Each of the second electrical signals is shifted in time with respect to at least one other second electrical signal. And each of the second electrical signals is electrically isolated from all other second electrical signals.

Optionally, in the instances described in the previous paragraph, the first electrical signals are applied shifted in time to steer an electric field within the target region, and the second electrical signals are applied shifted in time to steer an electric field within the target region. And optionally, in these instances, each of the first electrical signals alternates between a positive polarity and a negative polarity, and each of the second electrical signals alternates between a positive polarity and a negative polarity.

### BRIEF DESCRIPTION OF THE DRAWINGS

FIG. 1 is a block diagram of the prior art Optune® system for delivering TTFields.

FIG. 2 is a more detailed view of the transducer arrays of FIG. 1.

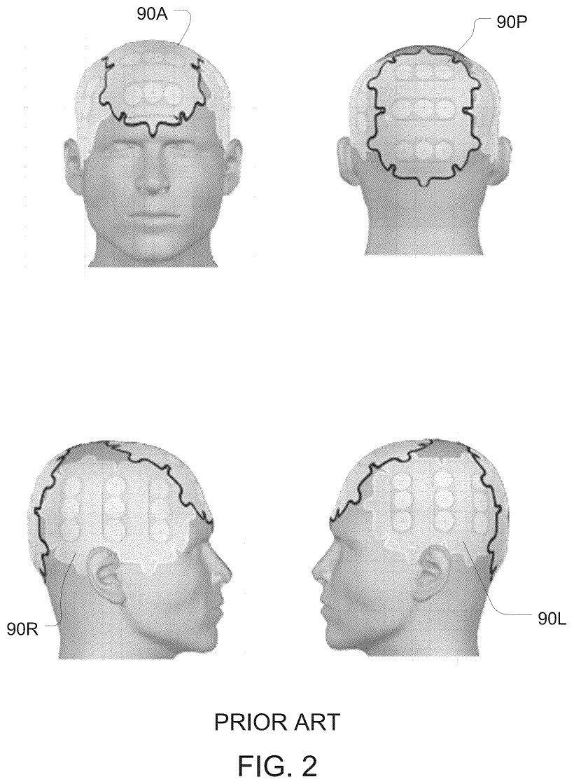

FIG. 3 depicts a set of four transducer arrays positioned on a patient's skin near a tumor.

FIG. 4 is a block diagram of a system for applying electrical signals to the electrode elements in the transducer arrays depicted in FIG. 3.

FIG. 5 depicts controlling the phases of signals that are generated by a set of signal generators to produce a specific set of output signals.

Various embodiments are described in detail below with reference to the accompanying drawings, wherein like reference numerals represent like elements.

### DESCRIPTION OF THE PREFERRED EMBODIMENTS

This application discloses an alternative approach for shifting the paths of the alternating electric fields that travel through the relevant body part. And this approach can, in many situations, increase in field strength in the target region beyond the level that can be attained using the prior art approach of shifting the positions of the transducer arrays.

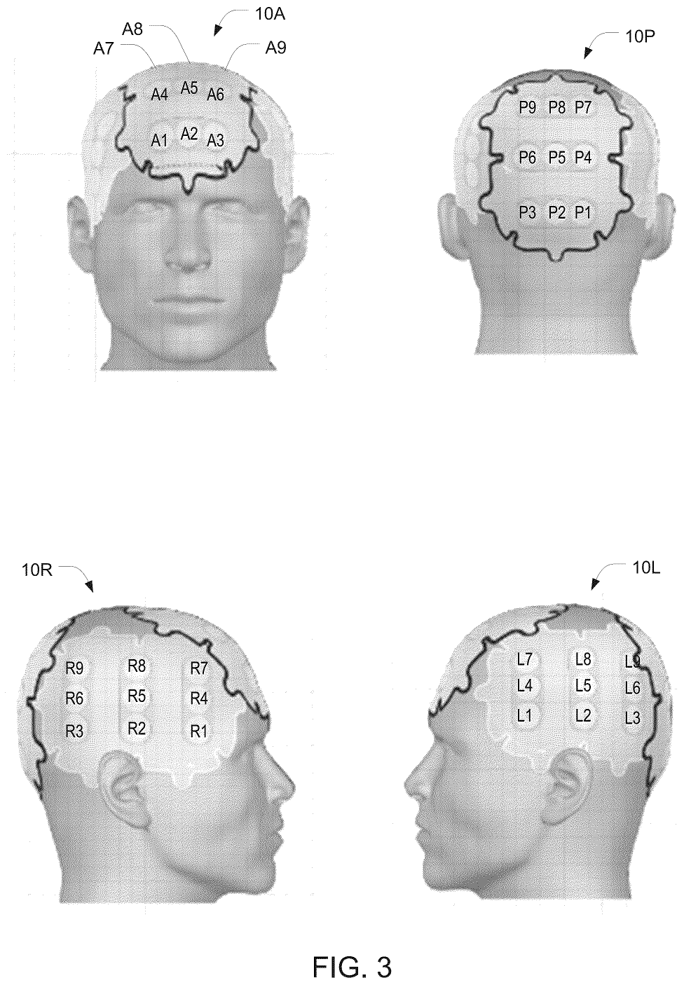

More specifically, instead of using a single signal generator to apply a signal between all of the electrode elements on one side of the target region and all of the electrode elements on the opposite side of the target region (as in the prior art), the embodiments described below in connection with FIGS. 3-5 use at least eight electrically isolated signal generators to apply electrical signals between respective electrode elements on one side of the target region and respective electrode elements on the opposite side of the target region. This replaces the prior art's single wide-cross-section electric field with eight independently controllable electric fields, each of which has a relatively narrow cross-section. Using these relatively narrow cross-section fields, either alone or in combination, can improve aiming of the electric field, which can in turn increase the field strength in the target region. And optionally, activation of these relatively narrow cross-section fields can be shifted in time (e.g., phase-shifted) to achieve steering of the overall electric field.

FIG. 3 depicts a set of four transducer arrays 10 that are placed on the patient's skin near the tumor. The transducer arrays 10 are arranged in two pairs, with one pair of transducer arrays 10L, 10R positioned to the left and right of the tumor, and the other pair of transducer arrays 10A, 10P positioned anterior and posterior to the tumor. Each of the transducer arrays 10 is, in most respects, similar to the prior art Optune® transducer arrays 90 described above. But unlike the Optune® transducer arrays, all of the electrode elements on any given transducer array 10 are not wired together in series or in parallel. Instead, each of the electrode elements A1-A9 on the anterior transducer array 10A is provided with its own individual wire so it can be driven independently. And similarly, each of the electrode elements P1-P9, L1-L9, and R1-R9 on the posterior, left, and right arrays 10P, 10L, 10R is provided with its own respective individual wire so it can be driven independently.

Providing an individual wire for each electrode element on each of the transducer arrays 10 enables an individualized signal to be applied it to each of the electrode elements on each transducer array independently, without affecting the other electrode elements on the transducer array. And the benefits provided by this arrangement are described below.

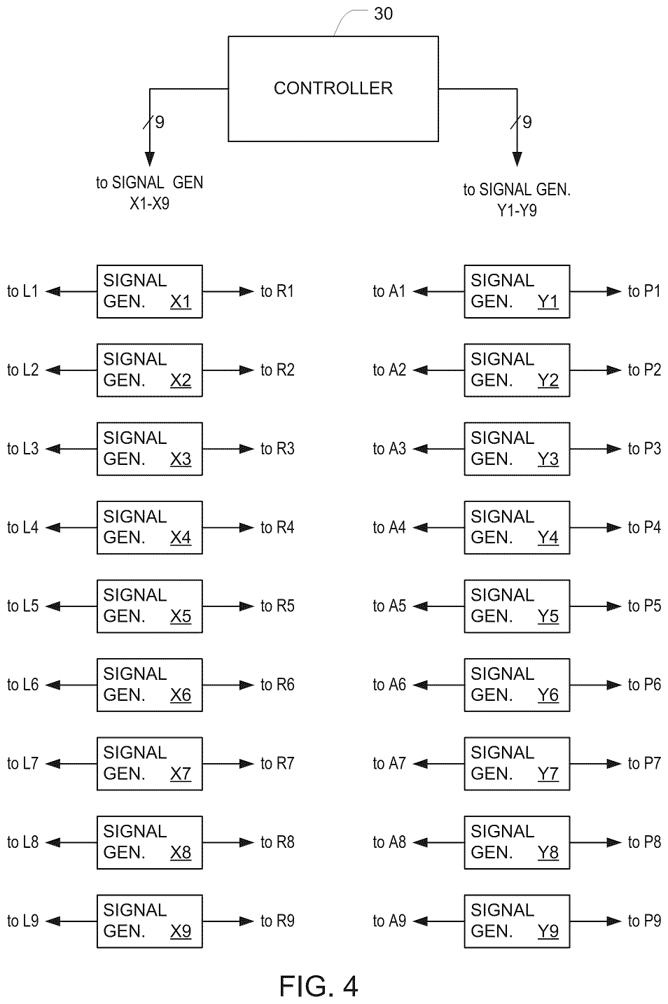

FIG. 4 is a block diagram of a system for applying electrical signals to the electrode elements A1-A9, P1-P9, L1-L9, and R1-R9 depicted in FIG. 3. The system in the illustrated embodiment includes nine first signal generators X1-X9. And each of these first signal generators X1-X9 is wired up to apply a signal between a respective single one of the electrode elements L1-L9 on the left array 10L and a respective single one of the electrode elements R1-R9 on the right array 10R. More specifically, signal generator Xi is wired to apply a signal between electrode element Li and electrode element Ri, where i is an integer between 1 and 9. In some embodiments, the output of any given one of the first signal generators Xi can be either positive (i.e., where the respective L terminal is the anode and the respective R terminal is the cathode), negative (i.e. where the respective L terminal is the cathode and the respective R terminal is the anode), or off (i.e. where no pulse is generated).

Each of the first signal generators X1-X9 is electrically isolated from all the other signal generators. As a result, the signal that signal generator X1 applies between electrode element L1 and electrode element R1 will not affect the signals that are applied to any of the other electrode elements L2-L9, R2-R9, A1-A9, or P1-P9. A similar situation exists for all the other first signal generators X2-X9, so that the signals that those signal generators send to their respective electrode elements will not affect the signals that are applied to any of the other electrode elements.

Each of the first signal generators X1-X9 has a respective first control input, and each of the first signal generators X1-X9 is configured to, in response to a respective first control signal that arrives at the respective first control input, apply an electrical signal between a respective one of the electrode elements L1-L9 and a respective one of the electrode elements R1-R9. A controller 30 is programmed to generate each of these first control signals.

Each of the first signal generators X1-X9 can be configured to, (a) in response to a respective first control signal that arrives at the respective first control input, apply a positive electrical signal between the respective one of the electrode elements L1-L9 and the respective one of the electrode elements R1-R9, and (b) in response to a respective second control signal that arrives at the respective second control input, apply a negative electrical signal between the respective one of the electrode elements L1-L9 and the respective one of the electrode elements R1-R9. Note that the use of the identifiers (a) and (b) does not imply that (a) must precede (b) in time.

When the first signal generators are implemented as described in the previous paragraph, the controller 30 can be programmed to, for each of the first signal generators X1-X9, apply the respective first control signal and the respective second control signal at respective times in an alternating sequence so that the respective first signal generator will generate an output that alternates between a positive electrical signal and a negative electrical signal. Optionally, in these embodiments, the controller 30 can be further programmed to interpose a break in time between each first control signal and each second control signal.

The system in the illustrated embodiment also includes nine second signal generators Y1-Y9. And each of these second signal generators Y1-Y9 is wired up to apply a signal between a respective single one of the electrode elements A1-A9 on the anterior array 10A and a respective single one of the electrode elements P1-P9 on the posterior array 10P. More specifically, signal generator Yj is wired to apply a signal between electrode element Aj and electrode element Pj, where j is an integer between 1 and 9. In some embodiments, the output of any given one of the second signal generators Yj can be either positive (i.e., where the respective A terminal is the anode and the respective P terminal is the cathode), negative (i.e. where the respective A terminal is the cathode and the respective P terminal is the anode), or off (i.e. where no pulse is generated).

Each of the second signal generators Y1-Y9 is electrically isolated from all the other signal generators. As a result, the signal that signal generator Y1 applies between electrode element A1 and electrode element P1 will not affect the signals that are applied to any of the other electrode elements A2-A9, P2-P9, L1-L9, or R1-R9. A similar situation exists for all the other second signal generators Y2-Y9, so that the signals that those signal generators send to their respective electrode elements will not affect the signals that are applied to any of the other electrode elements.

Each of the second signal generators Y1-Y9 has a respective second control input, and each of the second signal generators Y1-Y9 is configured to, in response to a respective second control signal that arrives at the respective second control input, apply an electrical signal between a respective one of the electrode elements A1-A9 and a respective one of the electrode elements P1-P9. The controller 30 is further programmed to generate each of these second control signals. Operation of the second signal generators Y1-Y9 is similar to the operation of the first signal generators X1-X9 described above.

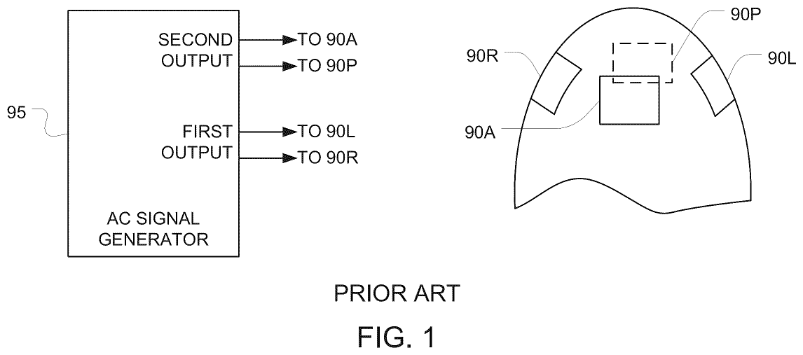

In the FIG. 1 prior art system, where all of the electrode elements on the left transducer array 90L are wired together in series or in parallel, and all of the electrode elements on the right transducer array 90R are wired together in series or parallel, a single electric field travels from all the electrode elements on the left transducer array 90L to all of the electrode elements on the right transducer array 90R. And a single electric field travels from all the electrode elements on the anterior transducer array 90A to all of the electrode elements on the posterior transducer array 90P. The resulting electric fields will therefore have comparatively large cross-sections, and can therefore be analogized to floodlights.

In contrast, in the FIG. 3/4 embodiment, one electric field travels from electrode element L1 to electrode element R1 when signal generator X1 is activated, a second electric field travels from electrode element L2 to electrode element R2 when signal generator X2 is activated, a third electric field travels from electrode element L3 to electrode element R3 when signal generator X3 is activated, etc. As a result, nine distinct electric fields can travel from respective electrode elements L1-L9 to respective electrode elements R1-R9, depending on which ones of the signal generators X1-X9 are activated. When any given single one of the signal generators X1-X9 is activated, the resulting electric field will have a comparatively small cross-section and can therefore be analogized to a spotlight.

Notably, more than one of the signal generators X1-X9 can be activated simultaneously (e.g., any 2 at a time, any 3 at a time, etc., up to all 9 at a time). When all nine of the signal generators X1-X9 are activated simultaneously, the resulting electric field will have a cross-section that is similar to the electric field that was generated using the prior art transducer arrays 90L/90R. The latter situation can be analogized to the situation where the light from nine individual spotlights collectively illuminate the same area as a floodlight.

Similarly, one electric field travels from electrode element A1 to electrode element P1 when signal generator Y1 is activated, a second electric field travels from electrode element A2 to electrode element P2 when signal generator Y2 is activated, a third electric field travels from electrode element A3 to electrode element P3 when signal generator Y3 is activated, etc. As a result, nine distinct electric fields can travel from respective electrode elements A1-A9 to respective electrode elements P1-P9, depending on which ones of the signal generators Y1-Y9 are activated. When any given single one of the signal generators Y1-Y9 is activated, the resulting electric field will have a comparatively small cross-section and can therefore be analogized to a spotlight.

As described above in connection with the first signal generators X1-X9, more than one of the second signal generators Y1-Y9 can be activated simultaneously (e.g., any 2 at a time, any 3 at a time, etc., up to all 9 at a time). When all nine of the signal generators Y1-Y9 are activated simultaneously, the resulting electric field will have a cross-section that is similar to the electric field that was generated using the prior art transducer arrays 90A/90P. The latter situation can again be analogized to the situation where the light from nine individual spotlights collectively illuminate the same area as a floodlight.

Breaking the electric field up into individual narrower components as described above can be very useful for concentrating the electric field onto the target region. This can be analogized to the way that nine individually controllable spotlights can concentrate illumination onto a given target region more effectively than a single floodlight.

Above and beyond the added level of control that is provided by breaking the electric field up into individual narrower components in order to concentrate the electric field onto the target region, a further level of control over the electric field in the target region can be obtained by controlling the phases of the signals that are generated by the signal generators X1-X9 and Y1-Y9.

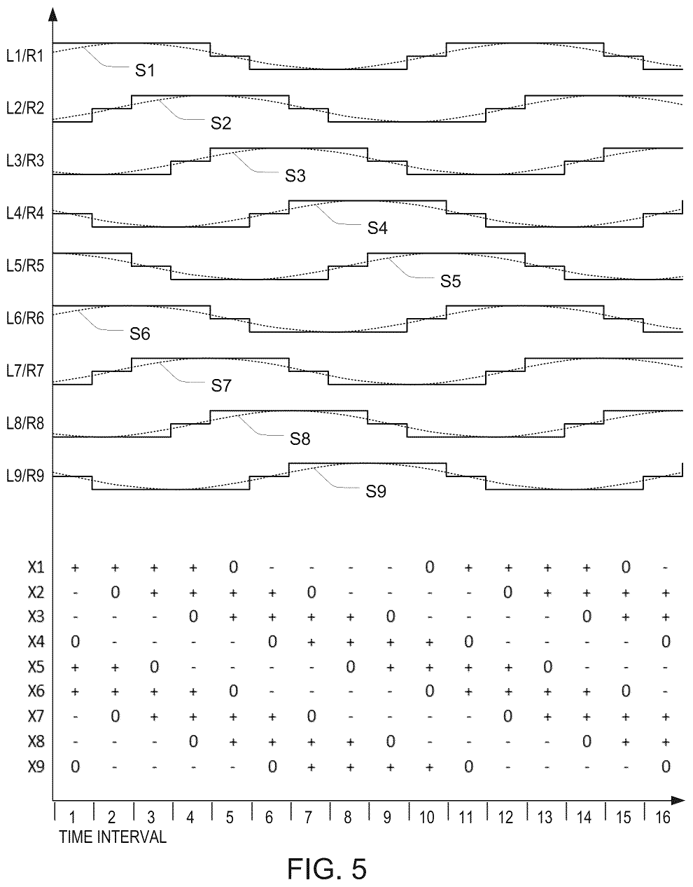

FIG. 5 depicts one example of how the controller 30 can control the phases of the signals that are generated by the signal generators X1-X9 to cause those signal generators to generate output signals that resemble a piecewise approximation to phase-shifted sinusoids S1-S9. More specifically, the controller 30 is programmed to generate control signals in a sequence that causes each of the signal generators X1-X9 to generate an output with a given waveform, so that the output of each of the signal generators X1-X9 is shifted in time with respect to the output of at least one other signal generator X1-X9. An example of the control signals that the controller 30 sends to the signal generator X1 are shown in the bottom half of FIG. 5, with a + representing a command to generate a positive signal, a zero representing a command to generate a zero output, and a − representing a command to generate a negative signal. And the outputs that the signal generators X1-X9 send to the respective sets of electrode elements L1/R1-L9/R9 are shown in the top half of FIG. 5.

When the signal generator X1 receives the +/−/0 control signals depicted in the top row of the bottom half of FIG. 5, the signal generator X1 will generate the digital signal depicted in the top row of the top half of FIG. 5. More specifically, signal generator X1 will (a) apply a positive signal to electrode elements L1 and R1 in time intervals 1-4; (b) apply no signal to those electrode elements in time interval 5; (c) apply a negative signal to those electrode elements in time intervals 6-9; (d) apply no signal to those electrode elements in time interval 10; (e) apply a positive signal to those electrode elements in time intervals 11-14; (e) apply no signal to those electrode elements in time interval 15; and (f) apply a negative signal to those electrode elements in time interval 16. And notably, an overlay of sinusoid S1 with the digital signal depicted in the top row of FIG. 5 makes it clear that this digital signal is a first-order piecewise approximation to sinusoid S1.

The controller 30 sends similar signals to the other signal generators X2-X9, but shifted in time with respect to the signal that is sent to the signal generator X1, as depicted in the bottom half of FIG. 5. The signal generators X2-X9 will respond by applying digital signals to their corresponding electrode element pairs L2/R2 through L9/R9 as depicted in the top half of FIG. 5. And these digital symbols resemble the digital signal described above for the electrode elements L1 and R1, except that they are shifted in time as depicted in the top half of FIG. 5. Here again, an overlay of sinusoids S2-S9 with the digital signals that are applied to the electrode element pairs L2/R2 through L9/R9 make it clear that each of these digital signals is a first-order piecewise approximation of a respective one of the sinusoids S2-S9, and that each of those sinusoids (except S6) is phase-shifted with respect to sinusoid S1.

Optionally, the controller 30 can be programmed to control the shifting in time to steer the electric field within the target region. Under certain conditions, especially when the number of signal generators is large, the ability to generate phase-shifted sinusoids can be used to control the direction of the electric field by implementing beam-steering techniques similar to those used in phased-array radar systems. These techniques can therefore be used to direct the electric field to a target region with an even finer degree of control that can be achieved using the spotlight-shaped electric fields described above in connection with FIG. 3-4.

The operation of the other set of signal generators Y1-Y9 is similar to the operation of the first set of signal generators X1-X9 described above.

Note that while the FIG. 3/4 embodiment is described above in the context of an example that includes nine electrode elements A1-A9, P1-P9, L1-L9, and R1-R9 on each of the transducer arrays 10, the number of electrode elements on each of the transducer arrays 10 can vary (e.g., between 8 and 64). Similarly, instead of the depicted nine first signal generators X1-X9 and nine second signal generators Y1-Y9, the number of first signal generators X can vary, as long as there are at least eight (e.g., between 8 and 64). The number of second signal generators Y can also vary, as long as there are at least eight (e.g., between 8 and 64). The number of signal generators will typically match the number of electrode elements in the corresponding transducer arrays 10.

Finally, in some anatomic locations, only a single pair of transducer arrays 10 is used. In these embodiments, one bank of signal generators (e.g., Y1-Y9 in FIG. 4) and two of the transducer arrays (e.g., 10A and 10P) are omitted.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an electric field to a target region using at least eight first electrode elements positioned on a first side of the target region and at least eight second electrode elements positioned on a second side of the target region that is opposite to the first side of the target region, the apparatus comprising:
at least eight electrically isolated first signal generators, wherein each of the first signal generators has a respective first control input, and wherein each of the first signal generators is configured to, in response to a respective first control signal that arrives at the respective first control input, apply an electrical signal between a respective one of the at least eight first electrode elements and a respective one of the at least eight second electrode elements; and
a controller that is programmed to generate each of the first control signals.

2. The apparatus of claim 1, wherein each of the first signal generators is configured to, (a) in response to a respective first control signal that arrives at the respective first control input, apply a positive electrical signal between the respective one of the at least eight first electrode elements and the respective one of the at least eight second electrode elements, and (b) in response to a respective second control signal that arrives at the respective second control input, apply a negative electrical signal between the respective one of the at least eight first electrode elements and the respective one of the at least eight second electrode elements.

3. The apparatus of claim 2, wherein the controller is further programmed to, for each of the first signal generators, apply the respective first control signal and the respective second control signal at respective times in an alternating sequence so that the respective first signal generator will generate an output that alternates between a positive electrical signal and a negative electrical signal.

4. The apparatus of claim 3, wherein the controller is further programmed to interpose a break in time between each first control signal and each second control signal.

5. The apparatus of claim 1, wherein the controller is further programmed to generate each of the first control signals in a sequence that causes each of the at least eight first signal generators to generate an output with a given waveform, wherein the output of each of the at least eight first signal generators is shifted in time with respect to an output of at least one other first signal generator.

6. The apparatus of claim 5, wherein the controller is further programmed to control the shifting in time to steer an electric field within the target region.

7. The apparatus of claim 5, wherein the controller is further programmed to generate each of the first control signals in a sequence that causes each of the at least eight first signal generators to generate an output that alternates between a positive electrical signal and a negative electrical signal.

8. The apparatus of claim 1, further comprising:
the at least eight first electrode elements; and
the at least eight second electrode elements.

9. The apparatus of claim 1, further comprising:
at least eight electrically isolated second signal generators, wherein each of the second signal generators has a respective second control input, and wherein each of the second signal generators is configured to, in response to a respective second control signal that arrives at the respective second control input, apply an electrical signal between a respective one of at least eight third electrode elements and a respective one of at least eight fourth electrode elements,
wherein the controller is further programmed to generate each of the second control signals.

10. The apparatus of claim 9, wherein the controller is further programmed to generate each of the first control signals in a sequence that causes each of the at least eight first signal generators to generate an output with a first given waveform, wherein the output of each of the at least eight first signal generators is shifted in time with respect to an output of at least one other first signal generator, and
wherein the controller is further programmed to generate each of the second control signals in a sequence that causes each of the at least eight second signal generators to generate an output with a second given waveform, wherein the output of each of the at least eight second signal generators is shifted in time with respect to an output of at least one other second signal generator.

11. A method for applying an electric field to a target region using at least eight first electrode elements positioned on a first side of the target region and at least eight second electrode elements positioned on a second side of the target region that is opposite to the first side of the target region, the method comprising:
applying respective first electrical signals between respective ones of the at least eight first electrode elements and respective ones of the at least eight second electrode elements, wherein the first electrical signals all have a given first waveform, wherein each of the first electrical signals is shifted in time with respect to at least one other first electrical signal, and wherein each of the first electrical signals is electrically isolated from all other first electrical signals.

12. The method of claim 11, wherein the first electrical signals are applied shifted in time to steer an electric field within the target region.

13. The method of claim 12, wherein each of the first electrical signals alternates between a positive polarity and a negative polarity.

14. The method of claim 11, further comprising:
positioning the at least eight first electrode elements on the first side of the target region; and
positioning the at least eight second electrode elements on the second side of the target region.

15. The method of claim 11, further comprising:
applying respective second electrical signals between respective ones of at least eight third electrode elements and respective ones of at least eight fourth electrode elements, wherein the second electrical signals all have a given second waveform, wherein each of the second electrical signals is shifted in time with respect to at least one other second electrical signal, and wherein each of the second electrical signals is electrically isolated from all other second electrical signals.

16. The method of claim 15, wherein the first electrical signals are applied shifted in time to steer an electric field within the target region, and
wherein the second electrical signals are applied shifted in time to steer an electric field within the target region.

17. The method of claim 16, wherein each of the first electrical signals alternates between a positive polarity and a negative polarity, and
wherein each of the second electrical signals alternates between a positive polarity and a negative polarity.
